(19) United States
(12) Patent Application Publication
(10) Pub. No.: US 2011/0301558 A1
(43) Pub. Date: Dec. 8, 2011

(54) Ostomy ring

(75) Inventor: Lorena Jean Vogel, Albuquerque, NM (US)

(73) Assignee: Lorena Jean Vogel, Albuquerque, NM (US)

(21) Appl. No.: 12/802,278

(22) Filed: Jun. 3, 2010

(51) Int. Cl. A61F 5/445

(52) U.S. Cl. 604/338

(57) ABSTRACT

By placing a surgical metal ring during on ostomy surgery the removal and replacement of ostomy bags will no longer require an adhesive that can cause irritation and sometimes not work.

This item is to be used for the improvement of life for ostomy patients. When a patient is to undergo an ostomy surgery a surgical metal ring may be placed in the epidermis during the surgery. An equal sized magnet can then be used for the placement of the ostomy bag over the skin without the use of adhesives. This will eliminate the problem of skin sensitivity and allow the bag to be placed even under difficult situations. This will also make it easier for anyone assisting someone with an ostomy bag to help them replace it without extensive training.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER LISTING COMPACT DISC APPENDIX

Not Applicable

### BACKGROUND OF THE INVENTION

This item is intended to ease the use of ostomy supplies for those in need of them. It seems that people with ostomies have difficulty with changing the bags and getting them to stay in place. Magnets have been used for a very long time and I believe that this invention would make many peoples lives easier.

### BRIEF SUMMARY OF THE INVENTION

Using a metal ring and a magnetic ring ostomy bags can be replaced easily.

### BRIEF DESCRIPTION OF THE SEVERAL VIEWS OF THE DRAWING

Not Applicable

### DETAILED DESCRIPTION OF THE INVENTION

A surgical metal ring and an equally sized magnet with a medium draw strength.

### DRAWINGS

Not Applicable

1. These items when used together will make it easier to remove and replace ostomy bags for those individuals who require the use of them.
